(19) United States
(12) Plant Patent Application Publication
(10) Pub. No.: US 2006/0288461 P1
(43) Pub. Date: Dec. 21, 2006

(54) CHRYSANTHEMUM PLANT NAMED 'MOZA THREE LPVES'

(50) CHRYSANTHEMUM MORIFOLIUM; CHRYSANTHEMUM

(76) Inventor: Leo Van Zanten, Oxnard, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/157,144

(22) Filed: Jun. 20, 2005

(51) Int. Cl. A01H 5/00

(52) U.S. Cl. PLT/286

(57) ABSTRACT

A new and distinct Chrysanthemum plant cultivar is disclosed, characterized by a daisy-type flower with a small disc and pink and white bi-colored ray florets. The plant naturally blooms during the last two weeks of September, with a floral longevity of about 26 days. The plant has a round, free branching growth habit and moderate vigor.

### LATIN NAME OF THE GENUS AND SPECIES

Chrysanthemum morifolium

### VARIETY DENOMINATION

‘MOZA THREE LPVES’

### BACKGROUND OF THE INVENTION

The new cultivar ‘MOZA THREE LPVES’ is a product of an induced mutation of parent Chrysanthemum plant variety ‘MOZA VES 02’ (U.S. Plant Pat. No. 14,633). The mutagenic treatment was performed in Carpenteria, Calif., in May, 2001, with a dose of 20 Gy (gamma rays) given to 750 vegetative cuttings. Additional cuttings were taken from the irradiated plants, so that the selection population included approximately 2,000 plants. The new cultivar ‘MOZA THREE LPVES’ was discovered and selected by Leo Van Zanten in a controlled environment in Oxnard, Calif., in January, 2002.

Asexual reproduction of the new cultivar ‘MOZA THREE LPVES’ by terminal cuttings and tissue culture was performed in Oxnard, Calif., and has shown that the unique features of this cultivar are stable and reproduced true to type on successive generations. Approximately five to six generations have come true to type from the original selection of the variety.

### SUMMARY OF THE INVENTION

The cultivar ‘MOZA THREE LPVES’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MOZA THREE LPVES.’ These characteristics in combination distinguish ‘MOZA THREE LPVES’ as a new and distinct Chrysanthemum cultivar:
1. Daisy-type flower.
2. Small disc.
3. Pink and white bi-colored ray florets.
4. Round growth habit.
5. Free-branching.
6. Floral longevity approximately 26 days.
7. Moderate plant vigor.
8. Naturally blooms during the last two weeks of September.

Plants of the new cultivar ‘MOZA THREE LPVES’ are similar to plants of the parent variety, ‘MOZA VES 02,’ in most horticultural characteristics. Plants of the new cultivar ‘MOZA THREE LPVES’ have ray florets that are a different color than the ray florets of the parent variety.

In comparison to the commercially available variety ‘MOZA CON 02’ (U.S. Plant Pat. No. 14,745), ‘MOZA THREE LPVES’ has a larger diameter, decorative-type flower with less ray florets. Additionally, the new variety naturally blooms approximately two weeks later than the comparable variety ‘MOZA CON 02’

In comparison to the commercially available variety ‘MOZA SI 01’ (U.S. Plant Pat. No. 14,772), ‘MOZA THREE LPVES’ has less ray florets, a smaller disc, and less disc florets. Additionally, the ray floret apex of the new variety is retuse, in contrast to an obtuse ray floret apex in the comparable variety ‘MOZA SI 01.’ Further, the new variety ‘MOZA THREE LPVES’ has a more freely branching, flexible plant habit than the comparable variety.

### BRIEF DESCRIPTION OF THE PHOTOGRAPH

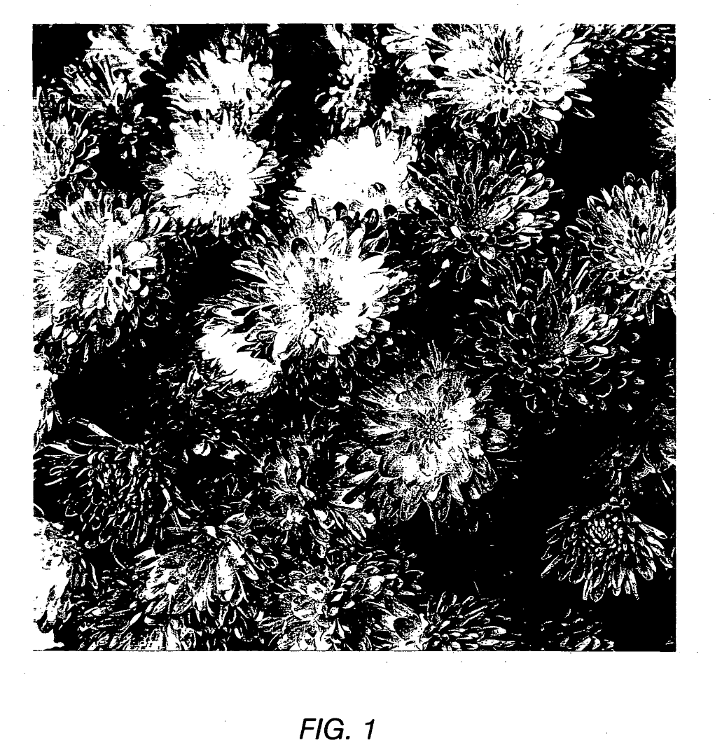

The accompanying photograph in FIG. 1 illustrates in full color a typical plant of ‘MOZA THREE LPVES’ grown in a 2 gallon container. One cutting was used in the pot. The colors are as nearly true as is reasonably possible in a color representation of this type.

### DETAILED BOTANICAL DESCRIPTION

In the following description, color references are made to The Royal Horticultural Society Colour Chart except where general terms of ordinary dictionary significance are used. The following observations and measurements describe ‘MOZA THREE LPVES’ plants grown in a greenhouse in Oxnard, Ventura County, Calif., during the month of September 2003. The growing temperature ranged from about 12° C. to 15° C. at night to about 12° C. to 20° C. during the day. Measurements and numerical values represent averages of typical flowering types, and are believed to apply to plants of the variety grown under similar conditions of soil and climate elsewhere:
Botanical classification: Chrysanthemum morifolium ‘MOZA THREE LPVES.’
Commercial classification: Garden-type Chrysanthemum.
Propagation:
Time to rooting: 7 to 14 days at approximately 21° C.
Root description: Fine, fibrous.
Plant:
Growth habit: Mounding herbaceous perennial.
Height: Approximately 18 cm.
Plant spread: Approximately 16 cm.
Growth rate: Moderate.
Branching characteristics: Free branching.
Length of lateral branches: Approximately 14 cm.
Number of leaves per lateral branch: Approximately 10.
Age of plant described: Approximately 50 days from a rooted cutting.
Foliage:
Leaf:
   Arrangement.—Alternate.
   Average length.—Approximately 4.2 cm.
   Average width.—Approximately 3.8 cm.
   Shape of blade.—Ovate.
   Apex.—Cuspidate.
   Base.—Attenuate.
   Attachment.—Stalked.
   Margin.—Palmately lobed and toothed.
   Texture of top surface.—Lightly pubescent.
   Texture of bottom surface.—Lightly pubescent.
   Leaf internode length.—Approximately 2.9 cm.
   Color.—Young foliage upper side: Near Green 137A. Young foliage under side: Near Green 138A. Mature foliage upper side: Near Green 137A. Mature foliage under side: Near Green 137A.
   Venation.—Type: Palmately net. Venation color upper side: Near Yellow-green 146A. Venation color under side: Near Yellow-green 146B.
Petiole:
   Average length.—Approximately 1.0 cm.
   Color.—Near Yellow-green 146A.
   Diameter.—Approximately 0.2 cm.
Bloom:
Flower:
   Flowering habit.—Induced by darkness period greater than approximately 13.5 hours, approximately 45 days of appropriate day length required to induce and develop blooms.
   Form.—Daisy.
   Natural flowering season.—Approximately the last 2 weeks of September.
   Number of flowers per lateral branch.—Approximately 12.
   Diameter.—Approximately 3.9 cm.
   Disc diameter.—Approximately 0.3 cm.
   Depth.—Approximately 1.5 cm.
   Longevity on plant.—Approximately 27 days.
   Persistence.—Persistent.
Ray florets:
   Appearance.—Matte.
   Texture.—Smooth.
   Average number of ray florets per flower.—Approximately 75.
   Shape.—Oblanceolate.
   Aspect.—Flat.
   Margin.—Entire.
   Apex.—Retuse.
   Length.—Approximately 1.5 cm.
   Width.—Approximately 0.5 cm.
   Color.—Upper surface at first opening: Near White 155D with near Red-purple 63D at apex. Upper surface at maturity: Near White 155D for lower 70%, near Red-purple 64D at upper 30%. Upper surface at fading: Near White 155D for lower 30%, near Red-purple 64D at upper 70%. Under surface at first opening: Near Yellow 8D. Under surface at maturity: Near White 155D for lower 30%, near Red 56B at upper 70%. Under surface at fading: Near Red 55B with streaks of near White 155D.
Disc florets:
   Appearance.—Shiny.
   Texture.—Smooth.
   Average number of disc florets per disc.—Approximately 60.
   Shape.—Cylindric.
   Apex.—Obtuse.
   Average length.—Approximately 1 cm.
   Average width.—Approximately 0.1 cm.
   Color.—At first opening: Near Yellow-green 154B. At maturity: Near Yellow 7B. At fading: Near Yellow-orange 20A.
Peduncle:
   Length.—At terminal end (shortest): Approximately 3.5 cm. At lateral end (longest). Approximately 6.5 cm.
   Angle to stem.—Acute.
   Strength.—Moderate.
   Color.—Near Green 137A.
   Habit.—Upright.
   Diameter.—Approximately 0.2 cm.
   Surface texture.—Lightly pubescent.
Flower bud:
   Length.—Approximately 0.8 cm.
   Diameter.—Approximately 0.8 cm.
   Form.—Globular.
   Color.—Near Yellow 8D.
Involucral bracts (phyllaries):
   Appearance.—Matte.
   Texture.—Lighly pubescent.
   Number.—Approximately 20.
   Shape.—Oblanceolate.
   Margin.—Entire.
   Apex.—Acute.
   Length.—Approximately 0.8 cm.
   Width.—Approximately 0.7 cm.
   Color.—Upper side: Near Green 137A. Under side: Near Green 137A.
Reproductive organs:
Ray florets:
   Number of pistils per floret.—1.
   Stigma shape.—2 branched.
   Stigma color.—Near Yellow 2C.
   Style color.—Near Yellow 2D.
   Style length.—Approximately 0.8 cm.
   Stamens.—Absent.
Disc forets:
   Number of pistils per floret.—1.
   Stigma shape.—Two-branched.
   Stigma color.—Near Yellow 7A.
   Style length.—Appoximately 0.3 cm.
   Style color.—Near Yellow 2D.
   Number of stamens per floret.—5.
   Anther shape.—Tubular.
   Anther color.—Near Yellow 13A.
   Pollen color.—Near Yellow-orange 16A.
Other characteristics:
Disease resistance: Neither resistance nor susceptibility to diseases or pests has been observed in this variety.
Drought tolerance/cold tolerance: Flowering plants are hardy to a low temperature of about −2° C. Non-flowering plants are hardy in the approximate range of 3° C. to −6° C., depending upon duration of cold an amount of moisture in the soil. With adequate water plants are hardy to a high temperature of 45° C.
Fruit/seed production.—Commercially, this plant is not used or observed in a stage wherein seeds would be produced. Therefore, seed production has not been observed.

What is claimed is:

1. A new and distinct cultivar of Chrysanthemum plant named ‘MOZA THREE LPVES’ as herein illustrated and described.
